(19) United States
(12) Patent Application Publication
(10) Pub. No.: US 2019/0059475 A1
(43) Pub. Date: Feb. 28, 2019

(54) Glove Roll

(71) Applicant: Alysa Ashton Amy, Waterman, IL (US)

(72) Inventor: Alysa Ashton Amy, Waterman, IL (US)

(21) Appl. No.: 16/106,709

(22) Filed: Aug. 21, 2018

Related U.S. Application Data
(60) Provisional application No. 62/548,975, filed on Aug. 23, 2017.

(51) Int. Cl. A41D 19/00; A61B 42/40

(52) U.S. Cl. CPC A41D 19/0082; A41D 19/0003; A61B 42/40

(57) ABSTRACT

A roll of disposable, rubber gloves that are attached to a paper-like material via, but not limited to, an adhesive, heat or vacuum sealing. By producing one single sheet of the glove roll at a time, it gives the capability to sandwich two sheets of the glove roll together to create individual sections of pairs of gloves. Producing the glove roll this way allows for the guarantee of infection control. The user is only receiving one pair of gloves at a time, rather than reaching into a box of gloves and pulling out too many and possibly contaminating the remaining gloves and/or wasting the extra. The roll will be dispensed by a machine that is similar in fashion to a paper towel dispenser and available in an automatic or manual dispensing option. The roll will also be dispensed via a traditional cardboard glove box.

### FIELD OF INVENTION

The present invention relates to the production, dispensing and donning of disposable gloves and infection prevention in, but not limited to use in hospitals, doctor/dentist offices and other settings such as meat markets, tattoo parlors and veterinary clinics.

### BACKGROUND

Infection control and prevention is a top priority in our nations hospitals. Healthcare facilities spend millions of dollars each year in attempts to control and limit the spread of infection between not only patients but healthcare workers alike. One of the top ways of preventing the spread of infection is using rubber gloves to protect the patient and caregiver. The traditional use of rubber gloves in healthcare settings at this time is subpar, and I'll tell you why. When a user reaches into a traditional box of gloves, they run into 3 major) problems.

Contaminating the remaining gloves in the box when a user reaches in with soiled or un-sanitized hands.

Pulling out more gloves than necessary in which case, leads to wasting of the extra gloves.

Or, putting the extra gloves back into the box, leading to again, contaminated gloves.

### BRIEF SUMMARY OF THE INVENTION

The present invention seeks to provide a solution to these problems by providing individual pairs of gloves, on a dispensable roll. By manufacturing and producing a single pair of clean, disposable gloves on a material such as but not limited to, a roll of recycled, recyclable, biodegradable, paper material.

In a preferred embodiment of the invention, the machine that dispenses the gloves will have three separate sections in order to house the standard 3 sizes of gloves (small, medium and large). Also, it will preferably have an optional attachment for a hand sanitizing station, in order to fully maintain cleanliness and guarantee the control of the spread of infection or bacteria. Also, the machine will preferably be made from but not limited to, an antimicrobial plastic. The machine that dispenses the glove roll will preferably be available in a manual and/or automatic dispensing option.

Also in a preferred embodiment of the present invention, the glove roll will be available in a standard glove box. The glove roll will be placed inside of a traditional cardboard box with a larger opening at the mouth of the box for simple dispensing. B dispensing the glove roll from a traditional box, it will give the user the option of keeping existing glove mounting products such as a wire rack.

### BRIEF DESCRIPTION OF DRAWINGS

FIG. 1 or FRONT VIEW

Item labeled #1 depicts two gloves, stacked on top of one another.

Item labeled #2 depicts a roll upon which gloves are stored and the continuation of more gloves on the roll. (ex . . . paper towels or toilet paper).

Item labeled #3 depicts a perforated edge for simple tearing.

Item labeled #4 depicts the material in which the gloves are sandwiched between.

FIG. 2 or SIDE VIEW

Item labeled #1 depicts two gloves stacked on top of one another. The gloves are encased between two pieces of the paper-like material.

1 Item labeled #2 depicts the roll upon which the gloves are stored.

FIG. 3 or INNER WORKINGS/OPEN FACED PRODUCT

FIG. 3 depicts to individual gloves that are applied to the paper-like material with, but not limited to, a medical grade adhesive. Then, the two separate sections of paper are stacked on top of each other, sandwiching the gloves together. The paper may be, but not limited to, heat sealed, vacuum sealed or sealed with medical grade adhesive.

FIG. 4 or TRADITIONAL GLOVE BOX

FIG. 4 depicts the traditional glove box option of the present invention. Preferably, the glove roll will be placed inside of the box and it will be opened by tearing off a piece of perforated cardboard on the mouth of the box, similar to a box of tissue. The glove roll is then accessible for use.

FIG. 5 or MANUAL GLOVE DISPENSER

FIG. 5 depicts the manual glove dispenser. This dispenser works similarly to a manual paper towel dispenser. The glove roll, is loaded inside the machine and the end of the roll is fed through the bottom of the dispenser. The user is able to access the glove roll from the exposed 1 inch of paper that protrudes out the bottom of the dispenser. Each pair of gloves from the roll will separate from the next by the perforations in the paper between each pair.

FIG. 6 or AUTOMATIC GLOVE DISPENSER

FIG. 6 depicts the automatic glove dispenser. This dispenser will work similarly to an automatic paper towel dispenser. The glove roll is loaded inside the machine and the end of he roll is fed through the bottom of the dispenser. The user is able to access the glove roll by waving the hand in front of the motion detector which will activate the machine to dispense a pair of gloves from the roll. Each pair of gloves from the roll will separate from the next by the perforations in the paper between each pair.

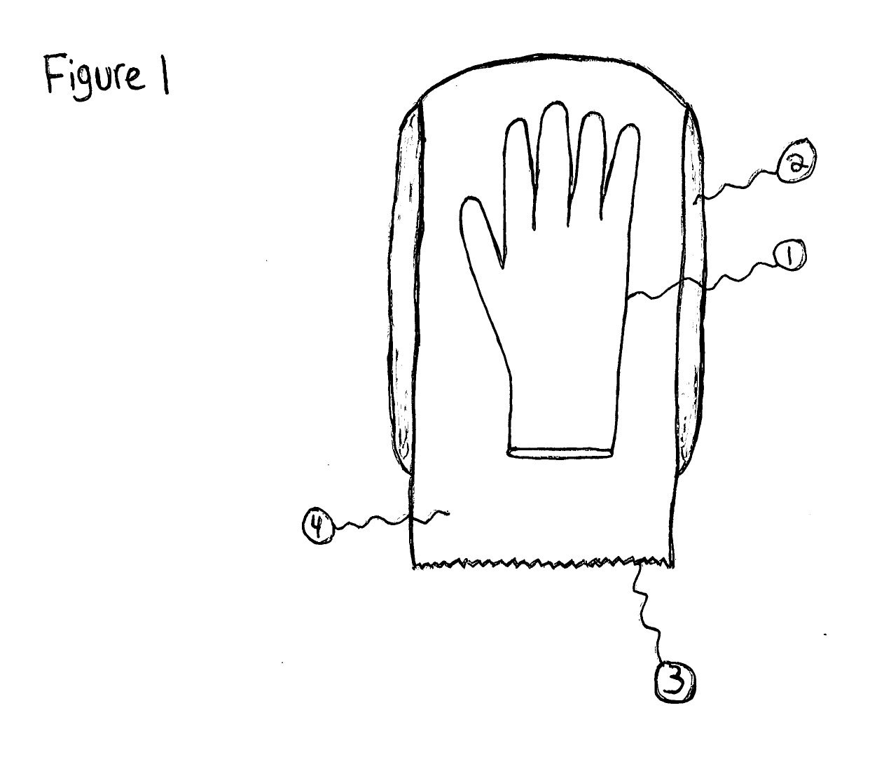

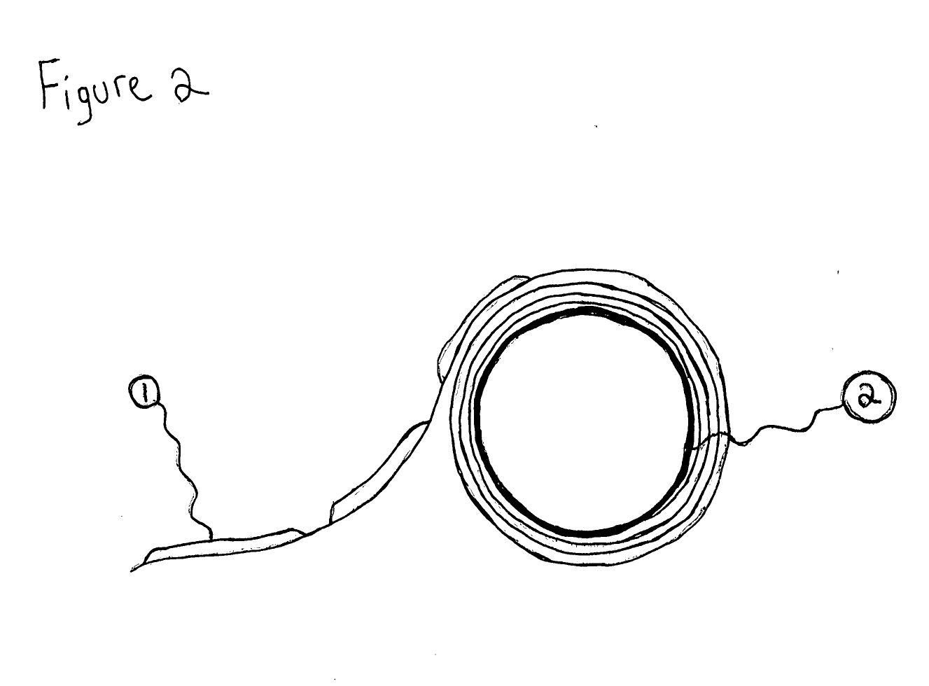

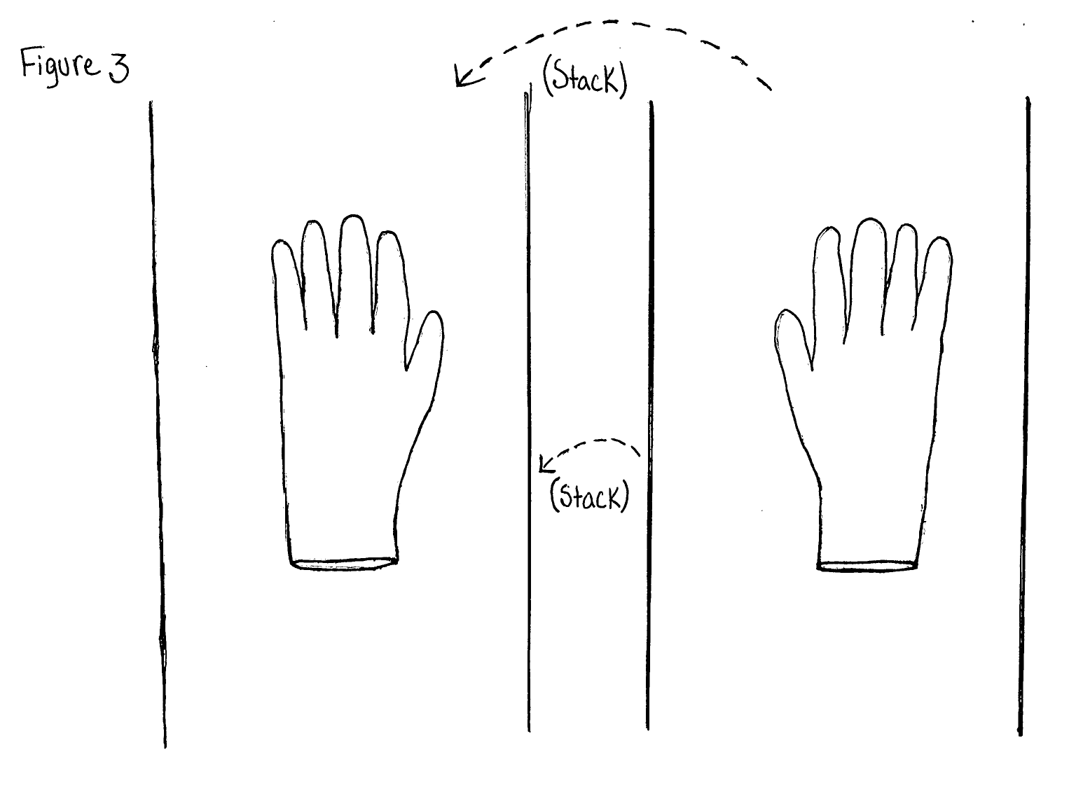

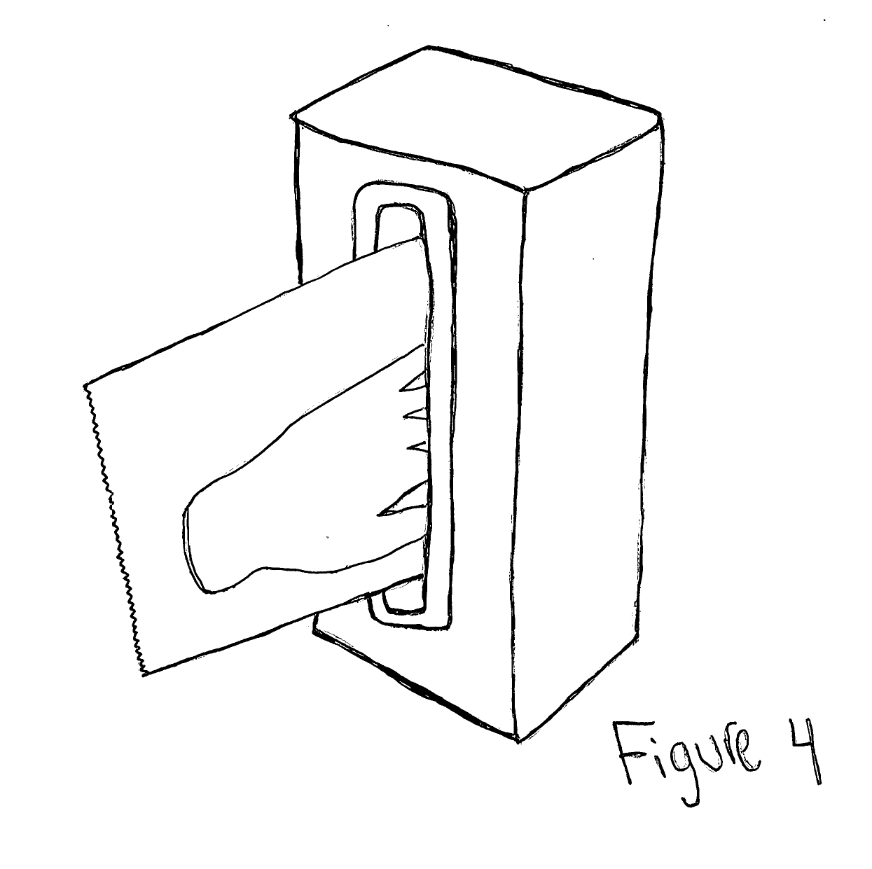

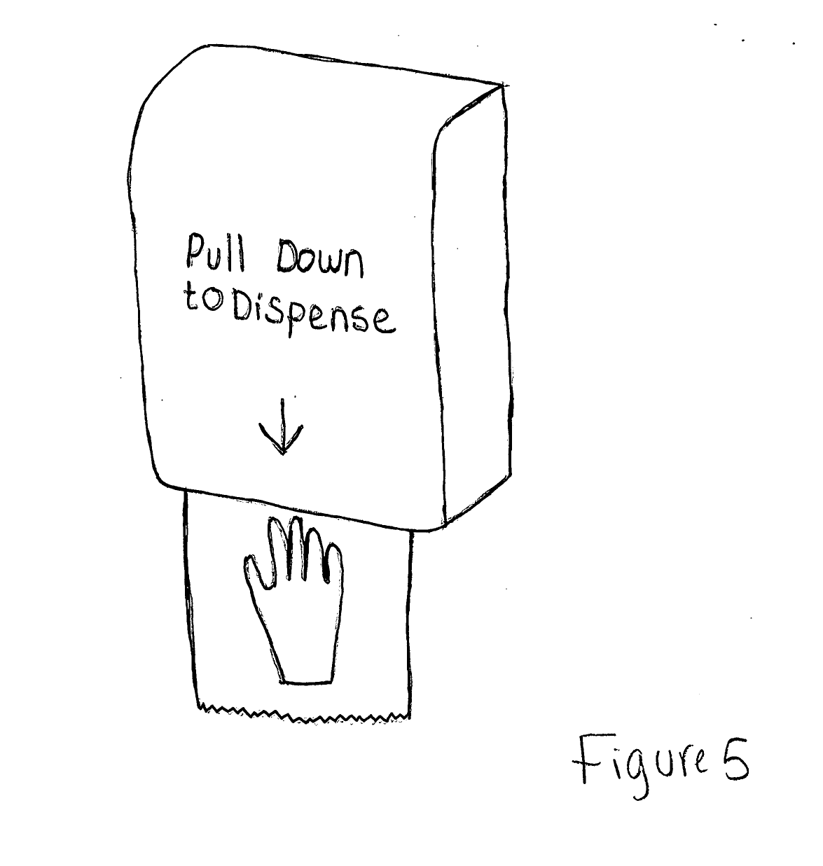

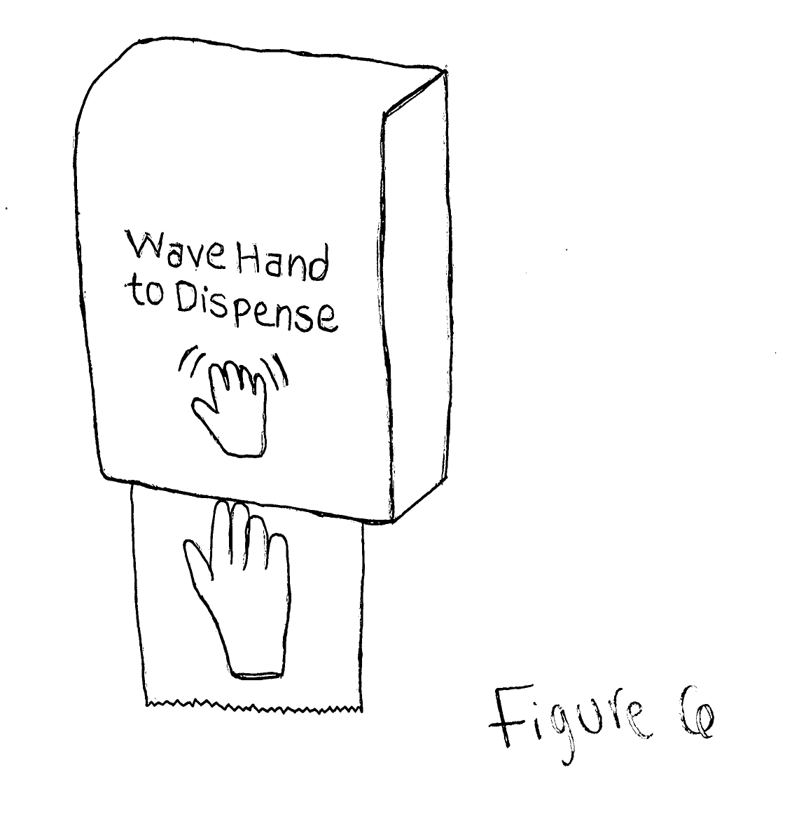

### DETAILED DESCRIPTION OF THE INVENTION

In a preferred embodiment of the present invention, the roll could be made from, but not limited to, recycled material. It could be impregnated with an antimicrobial solution and potentially be, but not limited to, covered with a gloss coat for easy removal of the gloves.

In a preferred embodiment of the present invention, the roll may be produced in an assembly line fashion. For example, the paper would be rolled out onto an assembly line. It could then be coated with, but not limited to, a medical grade adhesive. Then, workers would apply gloves in a continuous, uniform, single file line onto the paper.

In a preferred embodiment of the present invention, once two separate sections of the roll have been completed, they may be stacked on top of one another, sandwiching the gloves together.

In a preferred embodiment of the present invention, the two sections of paper can be, but not limited to, heat sealed, vacuum sealed or sealed with a medical grade adhesive.

In a preferred embodiment of the present invention, the roll should be perforated in between each set of gloves for simple tearing. The gloves will then be rolled up and stored on, but not limited to, a cardboard roll.

In a preferred embodiment of the present invention, the completed roll might be sealed and packaged in a, but not limited to, plastic wrap material or a tissue paper. Also, it may have an opening on the packaging that is accessible via the peeling of a sticker that would expose the opening.

In a preferred embodiment of the present invention, (IF USING THE MANUAL DISPENSER) the user would open the dispenser (which is similar in fashion to a paper towel dispenser), peel off the sticker that covers the opening to the roll, pull out the start of the roll and load it into the dispenser. Then the user will close the box.

In a preferred embodiment of the present invention, the user would first access the hand sanitization station. Then, the user would pull down on the exposed section of the roll, this motion would expose the single pair of gloves. Then, the user would tear off the gloves, peel the two sections apart revealing one pair of clean gloves. The user would then don the gloves and dispose of the remaining paper.

In a preferred embodiment of the present invention, (IF USING THE AUTOMATIC DISPENSER) the user would open the dispenser (which is similar in fashion to a paper towel dispenser), peel off the sticker that covers the opening to the roll, pull out the start of the roll and load it into the dispenser. Then the user will close the box.

In a preferred embodiment of the present invention, the user would wave his/her hand in front of the motion detector which would activate the machine to dispense a pair of gloves from the glove roll. Then, the user would tear off the gloves, peel the two sections apart revealing one pair of clean gloves. The user would then don the gloves and dispose of the remaining paper.

It shall be noted that a number of variations can be made to best suit the needs of the manufacturing company. The terms “such as” and “for example” are intended to highlight only some of the embodiments of the invention and they are not intended to be limiting. Some embodiments of the invention are drawn as an example and are not limited by the figures and/or the accompanying explanation or detailed description of the invention.

1. A roll of disposable gloves that is dispensed in a way that prevents the spread of infection nearly 100%.
